(19) United States
(12) Patent Application Publication
(10) Pub. No.: US 2025/0228529 A1
(43) Pub. Date: Jul. 17, 2025

(54) ULTRASONIC DIAGNOSIS APPARATUS, IMAGE PROCESSING APPARATUS, AND IMAGE PROCESSING METHOD

(71) Applicant: Canon Medical Systems Corporation, Otawara-shi (JP)

(72) Inventor: Itsuki KUGA, Utsunomiya (JP)

(73) Assignee: Canon Medical Systems Corporation, Otawara-shi (JP)

(21) Appl. No.: 19/023,614

(22) Filed: Jan. 16, 2025

(30) Foreign Application Priority Data
Jan. 16, 2024 (JP) 2024-004630

(51) Int. Cl. A61B 8/00; G06T 5/73; G06T 7/00

(52) U.S. Cl. CPC A61B 8/5223; A61B 8/465; A61B 8/488; A61B 8/5207; G06T 5/73; G06T 7/0012; G06T 2207/10136; G06T 2207/20192; G06T 2207/30104

(57) ABSTRACT

According to one embodiment, an ultrasonic diagnosis apparatus includes processing circuitry. The processing circuitry acquires three-dimensional Doppler data of an observation target, calculates a gradient of a surface of the observation target using a first element included in the Doppler data, generates a first rendering image of the observation target based on a second element included in the Doppler data, which is different from the first element, and generates a second rendering image considering shading based on the gradient and the first rendering image.

### CROSS-REFERENCE TO RELATED APPLICATIONS

This application is based upon and claims the benefit of priority from Japanese Patent Application No. 2024-004630, filed Jan. 16, 2024, the entire contents of which are incorporated herein by reference.

### FIELD

Embodiments described herein relate generally to an ultrasonic diagnosis apparatus, an image processing apparatus, and an image processing method.

### BACKGROUND

Conventionally, there is known a technique of displaying a bloodstream in a 3D (or 4D) color Doppler mode in an ultrasonic diagnosis apparatus. In general, an expected value to a 3D (three-dimensional) bloodstream image is considered to be higher for grasping the outer shape of a blood vessel than for grasping detailed velocity information of a bloodstream. This is because a three-dimensional bloodstream image can express the depth and the running direction of a bloodstream can be easily grasped.

A shading effect is sometimes applied to such a three-dimensional bloodstream image (rendering image). A rendering image considering shading further emphasizes the shape of a curved surface in a blood vessel, so the outer shape of the blood vessel can be easily grasped.

Calculation of shading uses, for example, information of the gradient (or normal) of the surface of a blood vessel. The gradient of the surface of the blood vessel is calculated from a value of data displayed in the Doppler mode. For example, in velocity display in the Doppler mode, the gradient is calculated from the velocity value of Doppler data. Also, for example, in power display in the Doppler mode, the gradient is calculated from the power value of Doppler data.

Specifically, the gradient can be calculated from a difference between Doppler data of six voxels surrounding a calculation target voxel (target voxel) at the center. Since the value of Doppler data is 0 in a space having no voxel, the gradient is large for a surface having no voxel adjacent to a target voxel. Thus, shading reflects the outer shape of the surface of a blood vessel.

In a velocity image, a boundary is sometimes generated between an area representing a flow toward a probe and an area representing a flow away from the probe. For a voxel near the boundary, the velocity value is small, so the gradient is large. Hence, shading emphasizes the boundary portion.

However, the emphasis of the boundary portion does not always represent the shape of the surface of a blood vessel near the boundary in the velocity image. As described above, the boundary can change depending on the position of the probe. A shading effect placing importance on the shape may not be applied in the velocity display in the Doppler mode.

As described above, data (for example, a velocity value and a power value) displayed in the Doppler mode and data used in shading calculation are conventionally common. Thus, there is only a few variations of shading.

### BRIEF DESCRIPTION OF THE DRAWINGS

FIG. 1 is a block diagram showing a configuration example of an ultrasonic diagnosis apparatus according to the first embodiment.

FIG. 2 is a flowchart showing an example of the operation of processing circuitry that executes rendering image generation processing according to the first embodiment.

FIG. 3 is a block diagram for explaining the first concrete example of the rendering image generation processing according to the first embodiment.

FIG. 4 shows an example of a rendering image considering shading according to the first embodiment.

FIG. 5 is a block diagram for explaining the second concrete example of the rendering image generation processing according to the first embodiment.

FIG. 6 is a block diagram for explaining the third concrete example of the rendering image generation processing according to the first embodiment.

FIG. 7 is a block diagram for explaining the fourth concrete example of the rendering image generation processing according to the first embodiment.

FIG. 8 is a view exemplifying a display image for which the rendering image generation processing according to the first embodiment is set.

FIG. 9 is a block diagram showing a configuration example of an ultrasonic diagnosis apparatus according to the second embodiment.

FIG. 10 is a flowchart showing an example of the operation of processing circuitry that executes rendering image generation processing according to the second embodiment.

FIG. 11 is a block diagram for explaining the first concrete example of the rendering image generation processing according to the second embodiment.

FIG. 12 is a block diagram for explaining the second concrete example of the rendering image generation processing according to the second embodiment.

FIG. 13 is a block diagram showing a configuration example of an information processing apparatus according to the third embodiment.

FIG. 14 is a block diagram showing a configuration example of an information processing apparatus according to the fourth embodiment.

FIG. 15 shows an example of a rendering image not considering shading.

FIG. 16 is a view of a conventional rendering image considering shading.

### DETAILED DESCRIPTION

In general, according to one embodiment, an ultrasonic diagnosis apparatus includes processing circuitry. The processing circuitry acquires three-dimensional Doppler data of an observation target, calculates a gradient of a surface of the observation target using a first element included in the Doppler data, generates a first rendering image of the observation target based on a second element included in the Doppler data, which is different from the first element, and generates a second rendering image considering shading based on the gradient and the first rendering image.

Hereinafter, embodiments of the ultrasonic diagnosis apparatus will be explained in detail with reference to the accompanying drawings.

### First Embodiment

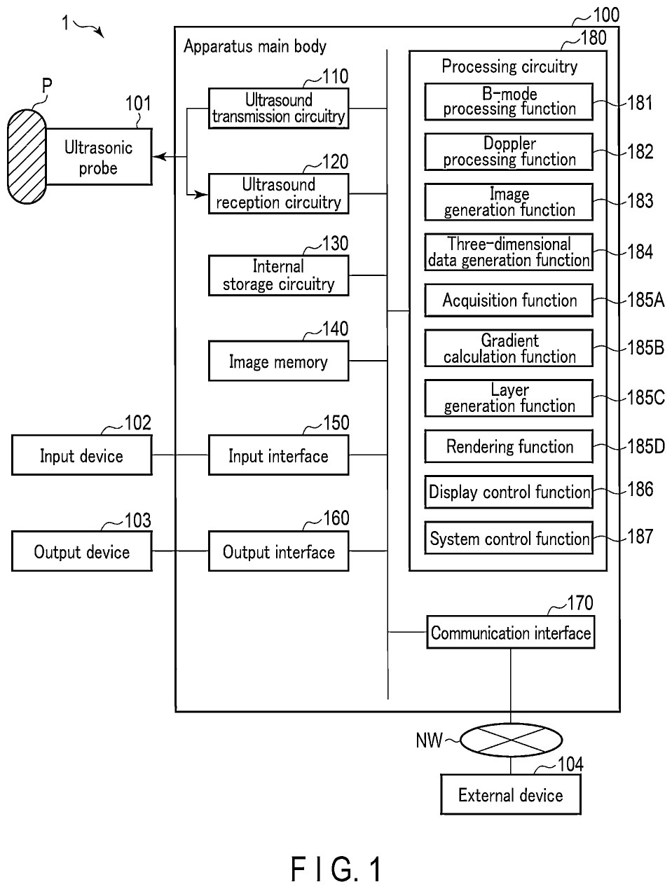

FIG. 1 is a block diagram showing a configuration example of an ultrasonic diagnosis apparatus according to the first embodiment. An ultrasonic diagnosis apparatus 1 shown in FIG. 1 includes an apparatus main body 100 and an ultrasonic probe 101. The apparatus main body 100 is connected to an input device 102 and an output device 103. The apparatus main body 100 is connected to an external device 104 via a network NW. The external device 104 is a server including, for example, picture archiving and communication systems (PACS).

The ultrasonic probe 101 performs ultrasonic scanning in a scan area of a living body P, which is a subject, under the control of, for example, the apparatus main body 100. The ultrasonic probe 101 includes, for example, a plurality of piezoelectric transducers, an acoustic lens, a matching layer provided between each of the piezoelectric transducers and the acoustic lens, and a backing material that prevents ultrasonic waves from propagating backward with respect to a direction of radiation from the piezoelectric transducers. The ultrasonic probe 101 is a two-dimensional array probe in which a plurality of ultrasonic transducers are aligned along, for example, a first element alignment direction (elevation direction) and a second element alignment direction (azimuth direction). The ultrasonic probe 101 is detachably connected to the apparatus main body 100. The ultrasonic probe 101 may be provided with a button which is to be depressed in an offset process or an operation for freezing ultrasonic images (freeze operation) or the like.

The piezoelectric transducers generate ultrasonic waves in response to a drive signal supplied from ultrasound transmission circuitry 110 included in the apparatus main body 100. An ultrasonic wave is thereby transmitted from the ultrasonic probe 101 to the living body P. When the ultrasonic wave is transmitted from the ultrasonic probe 101 to the living body P, the transmitted ultrasonic wave is sequentially reflected on the acoustic impedance discontinuous surfaces of the body tissue of the living body P, and is received as a reflection wave signal by the plurality of piezoelectric transducers. The amplitude of a received reflection wave signal depends on the difference in acoustic impedance on the discontinuous surfaces from which the ultrasonic wave is reflected. If the transmitted ultrasonic pulse is reflected from the surface of, for example, a moving bloodstream or a cardiac wall, the frequency of the resultant reflection wave signal will be shifted due to the Doppler effect, with the shift depending on the velocity component in the ultrasonic transmission direction of the moving object. The ultrasonic probe 101 receives the reflection wave signal from the living body P, and converts it into an electric signal.

FIG. 1 illustrates a connection relationship between the ultrasonic probe 101 and the apparatus main body 100. However, a plurality of ultrasonic probes may be connected to the apparatus main body 100. Which of the connected ultrasonic probes is to be used for the ultrasonic scanning can be selected freely through, for example, a software button on a touch panel (to be described later).

The apparatus main body 100 is an apparatus that generates an ultrasonic image based on the reflection wave signal received by the ultrasonic probe 101. The apparatus main body 100 includes the ultrasound transmission circuitry 110, ultrasound reception circuitry 120, internal storage circuitry 130, an image memory 140, an input interface 150, an output interface 160, a communication interface 170, and processing circuitry 180.

The ultrasound transmission circuitry 110 is a processor that supplies a drive signal to the ultrasonic probe 101. The ultrasound transmission circuitry 110 is realized by, for example, a trigger generation circuit, a delay circuit, and a pulsar circuit. The trigger generation circuit repeatedly generates rate pulses for forming transmission ultrasonic waves at a predetermined rate frequency. The delay circuit gives a delay time for each piezoelectric transducer to each rate pulse generated by the trigger generation circuit. This delay time is required to converge the ultrasonic wave generated from the ultrasonic probe into a beam and determine the transmission directivity. The pulsar circuit applies a drive signal (drive pulse) to a plurality of ultrasonic transducers of the ultrasonic probe 101 at the timing based on a rate pulse. By varying the delay time provided to each rate pulse by the delay circuit, the transmission direction from the surfaces of the piezoelectric transducers can be freely adjusted.

The ultrasound transmission circuitry 110 can freely change the output intensity of an ultrasonic wave by the drive signal. In the ultrasonic diagnosis apparatus, the influence of attenuation of ultrasonic waves in the living body P can be reduced by increasing the output intensity. In a reception time, a reflection wave signal with a high S/N ratio can be acquired by the ultrasonic diagnosis apparatus by reducing the influence of attenuation of ultrasonic waves.

Generally, when an ultrasonic wave propagates in the living body P, the intensity of ultrasonic vibrations (also called sound power) corresponding to the output intensity is attenuated. The attenuation of sound power is caused by absorption, scattering, reflection, etc. The degree of attenuation of the sound power depends on the frequency of the ultrasonic wave and the distance in radial direction of the ultrasonic wave. For example, the degree of attenuation is increased by increasing the frequency of the ultrasonic wave. Further, the degree of attenuation is increased as the distance in radial direction of the ultrasonic wave becomes longer.

The ultrasound reception circuitry 120 is a processor that performs various processes on the reflection wave signal received by the ultrasonic probe 101 and thereby generates a reception signal. The ultrasound reception circuitry 120 generates a reception signal based on the reflection wave signal of the ultrasonic wave acquired by the ultrasonic probe 101. Specifically, the ultrasound reception circuitry 120 is realized by, for example, a preamplifier, an A/D converter, a demodulator, and a beam former. The preamplifier performs gain correction processing by amplifying the reflection wave signal received by the ultrasonic probe 101 for each channel. The A/D converter converts the gain-corrected reflection wave signal into a digital signal. The demodulator demodulates the digital signal. The beam former provides the demodulated digital signal with delay time required to determine the reception directivity, and adds the digital signals to which delay time is provided. By the addition process of the beam former, a reception signal with an enhanced reflected component in a direction corresponding to the reception directivity is generated. Hereinafter, “the reflection wave signal of the ultrasonic wave” and “the reception signal” are collectively called “the echo signal”. Therefore, “the intensity of the reception signal” may be reworded to “the intensity of reflection of the echo signal (the echo reflection intensity)”.

The internal storage circuitry 130 includes a processor-readable storage medium, such as a magnetic storage medium, an optical storage medium, or a semiconductor memory. The internal storage circuitry 130 stores therein a program for realizing ultrasonic transmission/reception, a program relating to rendering image generation processing (to be described later), various data, etc. The programs and various data may be pre-stored in the internal storage circuitry 130. Alternatively, the programs and various data may be stored and distributed in a non-transitory storage medium, read from the non-transitory storage medium and installed in the internal storage circuitry 130.

The internal storage circuitry 130 stores B-mode image data, contrast image data, image data relating to bloodstream visualization, three-dimensional data, etc. generated by the processing circuitry 180, in accordance with an operation that is input via the input interface 150. The internal storage circuitry 130 can transfer the stored image data and three-dimensional data to the external device 104 or the like via the communication interface 170.

The internal storage circuitry 130 may be a drive etc. which reads and writes various types of information to and from a portable storage medium, such as a CD drive, a DVD drive, and a flash memory. The internal storage circuitry 130 may write the stored data onto a portable storage medium to store the data into the external device 104 by way of the portable storage medium.

The image memory 140 includes a processor-readable storage medium, such as a magnetic storage medium, an optical storage medium, or a semiconductor memory. The image memory 140 stores image data items corresponding to a plurality of frames immediately before a freeze operation input via the input interface 150. The image data stored in the image memory 140 is, for example, continuously displayed (cine-displayed). The image memory 140 may store not only the image data but also three-dimensional data.

The internal storage circuitry 130 and the image memory 140 are not necessarily implemented by independent storage devices. The internal storage circuitry 130 and the image memory 140 may be implemented by a single storage device. Each of the internal storage circuitry 130 and the image memory 140 may be implemented by a plurality of storage devices.

The input interface 150 receives various instructions from an operator through the input device 102. The input device 102 is, for example, a mouse, a keyboard, a panel switch, a slider switch, a trackball, a rotary encoder, an operation panel, or a touch panel. The input interface 150 is coupled to the processing circuitry 180 via a bus, for example, so that it can convert an operation instruction that is input by the operator, to an electric signal, and output the electric signal to the processing circuitry 180. The input interface 150 is not limited to physical operation components such as a mouse and a keyboard. For example, the input interface may include circuitry which receives an electric signal corresponding to an operation instruction input from an external input device provided independently from the ultrasonic diagnosis apparatus 1, and outputs the electric signal to the processing circuitry 180.

The output interface 160 is an interface to output, for example, the electric signal from the processing circuitry 180 to the output device 103. The output device 103 may be any display such as a liquid crystal display, an organic EL display, an LED display, a plasma display, or a CRT display. The output device 103 may be a touch-panel display that also serves as the input device 102. The output device 103 may also include a speaker configured to output a voice in addition to the display. The output interface 160 is connected to the processing circuitry 180, for example, via a bus, and outputs the electric signal coming from the processing circuitry 180 to the output device 103.

The communication interface 170 is connected to the external device 104 via, for example, the network NW, and performs data communication with the external device 104.

The processing circuitry 180 is a processor acting as a nerve center of the ultrasonic diagnosis apparatus 1, for example. The processing circuitry 180 executes the programs stored in the internal storage circuitry 130, thereby realizing the functions corresponding to the programs. The processing circuitry 180 includes, for example, a B-mode processing function 181, a Doppler processing function 182, an image generation function 183, a three-dimensional data generation function 184 functioning as a three-dimensional data generator, an acquisition function 185A functioning as an acquirer, a gradient calculation function 185B functioning as a gradient calculator, a layer generation function 185C functioning as a layer generator, a rendering function 185D functioning as a renderer, a display control function 186 functioning as a display controller, and a system control function 187.

The B-mode processing function 181 is a function of generating B-mode data based on the reception signals (echo signals) received from the ultrasound reception circuitry 120. By the B-mode processing function 181, the processing circuitry 180 performs an envelope detection process, a logarithmic compression process, or the like on a reception signal received from the ultrasound reception circuitry 120 to generate data (B-mode data) that expresses a signal intensity of the reception signal (echo reflection intensity) by a value of brightness (brightness value). The generated B-mode data is stored in a raw data memory (not shown in the drawings) as B-mode raw data on a two-dimensional ultrasonic scanning line (raster).

Furthermore, the processing circuitry 180 can perform harmonic imaging by the B-mode processing function 181. The harmonic imaging is an imaging method that utilizes not only a fundamental wave component but also a harmonic wave component (harmonic component) included in the reflection wave signal of the ultrasonic wave. The harmonic imaging includes, for example, a tissue harmonic imaging (THI) not using a contrast agent and a contrast harmonic imaging (CHI) using a contrast agent.

In the THI, a harmonic component can be extracted by using an amplitude modulation (AM) method, a phase modulation (PM) method, or an imaging method called an AMPM method, which is a combination of the AM method and the PM method.

With the AM method, the PM method, or the AMPM method, ultrasound wave transmission is performed more than once for a single scanning line, with different amplitudes and/or phases. Through the above processing, the ultrasound reception circuitry 120 generates a plurality of pieces of reflection wave data for each scanning line, and outputs the generated reflection wave data. The processing circuitry 180, by the B-mode processing function 181, performs addition and subtraction on the plurality of pieces of reflection wave data for each scanning line in accordance with a selected modulation method, thereby extracting a harmonic component. Furthermore, the processing circuitry 180 performs the envelope detection process or the like on the reflection wave data of the harmonic component, thereby generating B-mode data.

In the CHI, a harmonic component is extracted using, for example, a frequency filter. By the B-mode processing function 181, the processing circuitry 180 can separate reflection wave data (a harmonic component) whose reflection source is the contrast agent and reflection wave data (a fundamental wave component) whose reflection source is a living tissue in the living body P. As a result, the processing circuitry 180 can select a harmonic component from the contrast agent using a filter, thereby generating B-mode data to generate contrast image data.

The B-mode data used to generate contrast image data is data expressing an echo reflection intensity of the wave, whose reflection source is the contrast agent, as a brightness value. The processing circuitry 180 can also extract a fundamental wave component from the reflection wave data of the living body P, thereby generating B-mode data to generate living tissue image data.

The Doppler processing function 182 is a function of generating, by analyzing the frequencies of the reception signals received from the ultrasound reception circuitry 120, data (Doppler information) obtained by extracting motion information of a moving object in the region of interest (ROI) that is set in a scan area, based on the Doppler effect. The motion information of the moving object includes, for example, a velocity element, a turbulence (variance) element obtained by indexing variations of the velocity, and a power element obtained by differentiating the velocity. The generated Doppler information is stored in a raw data memory (not shown in the drawings) as Doppler raw data (also called Doppler data) on a two-dimensional ultrasonic scanning line.

Specifically, by the Doppler processing function 182, the processing circuitry 180 estimates as the motion information of the moving object, for example, a velocity value of the moving object, a turbulence value of the moving object, and a power value of the moving object signal at each of a plurality of sampling positions, and generates Doppler data indicating the estimated motion information. The moving object is, for example, a bloodstream, cardiac tissue such as a wall of a heart, or a contrast agent. The processing circuitry 180 according to the present embodiment estimates, by the Doppler processing function 182, a velocity value of a bloodstream, turbulence value of the bloodstream velocity, and a power value of a bloodstream signal as motion information of the bloodstream (bloodstream information) at each of the sampling positions, and generates Doppler data indicating the estimated bloodstream information. In other words, the Doppler data includes velocity data having a velocity value, turbulence data having a turbulence value, and power data having a power value.

The image generation function 183 is a function of generating B-mode image data based on the data generated by the B-mode processing function 181. By the image generation function 183, the processing circuitry 180 converts (scan-converts) a scanning line signal sequence of ultrasonic scanning into, for example, a scanning line signal sequence in a video format representatively used by a television, etc. to generate image data for display (display image data). Specifically, the processing circuitry 180 executes RAW-pixel conversion relative to B-mode RAW data stored in the RAW data memory, for example, executes coordinate conversion corresponding to the ultrasonic scan state by the ultrasonic probe 101, to generate two-dimensional B-mode image data (also referred to as ultrasonic image data) constituted by pixels. In other words, by the image generation function 183, the processing circuitry 180 generates a plurality of ultrasonic images (medical images) respectively corresponding to a plurality of consecutive frames by transmission and reception of ultrasonic waves.

The processing circuitry 180 performs, for example, a RAW-pixel conversion on the Doppler raw data stored in the raw data memory so as to generate Doppler image data in which bloodstream information is visualized. The Doppler image data is one of velocity image data, turbulence image data, and power image data, or image data obtained by a combination thereof. The processing circuitry 180 generates, as Doppler image data, color Doppler image data indicating bloodstream information in colors and gray-scale Doppler image data indicating a piece of bloodstream information as waveforms with a gray scale.

The three-dimensional data generation function 184 is a function of generating three-dimensional B-mode data (three-dimensional data) based on the reception signal received from the ultrasound reception circuitry 120. By the three-dimensional data generation function 184, the processing circuitry 180 allocates a brightness value to a voxel located in a three-dimensional space using the B-mode data generated by the B-mode processing function 181, thereby generating three-dimensional data. The three-dimensional data may be called volume data. Since the brightness value corresponds to the echo reflection intensity, it can be construed that the echo reflection intensity is allocated to the voxel of volume data. Therefore, “the brightness value of volume data” may be used in substantially the same meaning as “the echo reflection intensity”.

By the three-dimensional data generation function 184, the processing circuitry 180 according to this embodiment may generate Doppler data of three dimensions (three-dimensional Doppler data) by allocating Doppler data to a voxel located in a three-dimensional space.

The acquisition function 185A is a function of acquiring data about rendering image generation processing (to be described later). The data about rendering image generation processing includes, for example, a two-dimensional ultrasonic image, three-dimensional data, and a simple rendering image obtained by performing X-ray projection (full addition and projection) on three-dimensional Doppler data in a predetermined ray direction and rendering the data. A two-dimensional ultrasonic image will be simply called an ultrasonic image hereinafter. When an ultrasonic image and a simple rendering image are not discriminated, the two-dimensional ultrasonic image will be referred to as a two-dimensional image (2D image). The data about rendering image generation processing includes, for example, three-dimensional data and three-dimensional Doppler data. In this embodiment, the processing circuitry 180 acquires three-dimensional Doppler data of an observation target by the acquisition function 185A. The observation target in this embodiment is a blood flowing through a blood vessel.

The gradient calculation function 185B is a function of calculating the gradient of the surface of an observation target using an element (first element) included in three-dimensional Doppler data. By the gradient calculation function 185B, the processing circuitry 180 calculates the gradient of the surface of the observation target using the first element included in the three-dimensional Doppler data. When the observation target is a blood flowing through a blood vessel, the surface of the observation target substantially represents the surface of the blood vessel. Specifically, the processing circuitry 180 may calculate the gradient of the surface of the blood vessel using the power value of the Doppler data. The processing circuitry 180 may calculate the gradient of the surface of the blood vessel using the turbulence value of the Doppler data. Alternatively, the processing circuitry 180 may calculate the gradient of the surface of the blood vessel using the velocity value of the Doppler data.

The gradient can be calculated from values respectively included in six, front, rear, left, right, upper, and lower voxels with respect to a calculation target voxel (target voxel). Specifically, the processing circuitry 180 calculates the gradient based on a difference between the values of the front and rear voxels with respect to the target voxel, a difference between the values of the left and right voxels, and a difference between the values of the upper and lower voxels. The processing circuitry 180 calculates the gradient using, for example, equation (1) below:

$$\vec{n} ( x , y , z ) = \vert ∇ f ( x , y , z ) \vert = \vert ( \begin{matrix}\frac{\partial f}{\partial x} \\ \frac{\partial f}{\partial y} \\ \frac{\partial f}{\partial z}\end{matrix} ) \vert = \vert ( \begin{matrix}f ( x - 1 , y , z ) - f ( x + 1 , y , z ) \\ f ( x , y - 1 , z ) - f ( x , y + 1 , z ) \\ f ( x , y , z - 1 ) - f ( x , y , z + 1 )\end{matrix} ) \vert \qquad ( 1 )$$

In equation (1), f(x, y, z) represents a target voxel. At this time, the processing circuitry 180 calculates the gradient on the assumption that a value in a space having no data is 0. When there is no voxel adjacent to a target voxel, the target voxel can correspond to the surface of the observation target. Note that the processing circuitry 180 may specify in advance a voxel corresponding to the surface of an observation target, and calculate a gradient for only the specified voxel. Hereinafter, the processing circuitry 180 calculates the gradient of a voxel corresponding to the surface of an observation target.

The layer generation function 185C is a function of generating a shading layer to apply shading to a rendering image using the gradient of a voxel corresponding to the surface of an observation target. By the layer generation function 185C, the processing circuitry 180 calculates a shading using a known shading model, and generates a shading layer. The known shading model includes, for example, a diffuse specular shading model, a Phong reflection model, a Blinn-Phong shading model, and a specular highlight shading model. Note that the processing circuitry 180 may calculate a shading using a global illumination model accompanied by propagation of light.

The rendering function 185D is a function of generating a rendering image. The rendering images include, for example, a volume rendering image and a global illumination image. In this embodiment, a rendering image not taking a light source into account is defined as a volume rendering image, and a rendering image taking a light source into account is defined as a global illumination image.

The volume rendering image is obtained by volume-rendering volume data. In the volume rendering according to this embodiment, luminance and color displayed in each voxel is set in accordance with Doppler data allocated to the voxel of the volume data. Further, the volume rendering displays a projection image obtained by projection on the voxel from an arbitrary viewpoint.

On the other hand, the global illumination image is obtained by rendering processing by means of a photon map. For the rendering processing, for example, ray tracing is used. In this embodiment, the global illumination image may be generated as a final rendering image that is actually displayed.

By the rendering function 185D, the processing circuitry 180 generates the basic rendering image (first rendering image) of the observation target based on an element (second element) included in the three-dimensional Doppler data, which is different from the element (first element) used for gradient calculation. The first rendering image does not consider shading. Based on the calculated gradient and the first rendering image, the processing circuitry 180 generates the second rendering image considering shading.

Specifically, the processing circuitry 180 generates the second rendering image based on the first rendering image and a shading layer generated from the calculated gradient. More specifically, the processing circuitry 180 generates the second rendering image by superposing the shading layer on the first rendering image.

Note that the processing circuitry 180 may apply a predetermined filter to the first rendering image by the rendering function 185D. The predetermined filter is, for example, a nonlinear diffusion filter.

The display control function 186 is a function of causing a display as the output device 103 to display images based on various kinds of ultrasonic image data generated by the image generation function 183. Specifically, for example, by the display control function 186, the processing circuitry 180 controls the display of an image based on the B-mode image data, the Doppler image data, or image data including both generated by the image generation function 183.

More specifically, by the display control function 186, the processing circuitry 180 converts (scan-converts) a scanning line signal sequence of ultrasonic scanning into, for example, a scanning line signal sequence in a video format representatively used by television, etc. to generate display image data. The processing circuitry 180 may also perform various types of processing, such as correction of the dynamic range, brightness, contrast, and y curve, and an RGB conversion, on the display image data. The processing circuitry 180 may also add supplementary information, such as textual information of various parameters, a scale, or a body mark, to the display image data. The processing circuitry 180 may also generate a user interface (graphical user interface (GUI)) to allow the operator to input various instructions through the input device, and cause the display to display the GUI.

Furthermore, by the display control function 186, the processing circuitry 180 may display the rendering image generated by the rendering function 185D. The processing circuitry 180 may display a GUI related to setting of the rendering image together with the rendering image. The setting of the rendering image includes setting of parameters related to shading (shading parameters). The user can change the shading parameters displayed on the GUI to change a shading superposed on a basic rendering image into a desired display in real time. The user-changeable shading parameters are, for example, a shading gain (Shade Gain) indicating the intensity of shading and a shading type (Shade Type) indicating the type of shading. Note that the shading gain and the shading type will be described later.

The system control function 187 is a function of integrally controlling the overall operations of the ultrasonic diagnosis apparatus 1. For example, by the system control function 187, the processing circuitry 180 controls the ultrasound transmission circuitry 110 and the ultrasound reception circuitry 120 based on parameters relating to transmission and reception of ultrasonic waves.

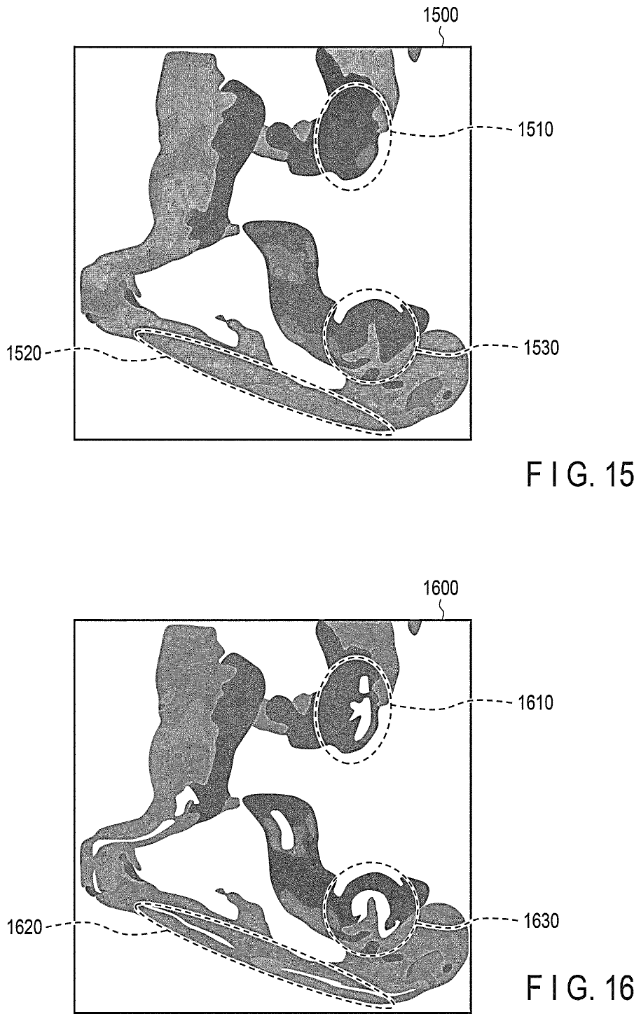

The configuration of the ultrasonic diagnosis apparatus according to the first embodiment has been described above. Next, a rendering image not considering shading and a conventional rendering image considering shading will be explained with reference to FIGS. 15 and 16.

FIG. 15 shows a rendering image not considering shading. A rendering image 1500 in FIG. 15 represents a blood vessel as an observation target by a three-dimensional velocity image. That is, the rendering image 1500 is a basic rendering image generated using velocity data included in Doppler data.

In the velocity image data according to this embodiment, the direction of a flow toward a probe is represented by a red color, and that of a flow away from the ultrasonic probe is represented by a blue color. In the rendering image 1500, a relatively dark color area 1510 corresponds to the blue color, and a relatively light color area 1520 corresponds to the red color. In an area 1530, the red color area penetrates part of the blue color area. No shading layer is superposed on the rendering image 1500, so when the user visually checks the rendering image 1500, the surface of the observation target seems flat.

FIG. 16 is a view of a conventional rendering image considering shading. A rendering image 1600 in FIG. 16 is obtained by superposing a shading layer generated using velocity data on the rendering image 1500 generated using velocity data.

In the rendering image 1600, an area 1610, an area 1620, and an area 1630 correspond to the area 1510, the area 1520, and the area 1530 in the rendering image 1500. Shadings (highlights) conforming to shapes of the observation target are applied to the areas 1610 and 1620. However, a shading conforming to the boundary between the blue color area and the red color area is recognized in the area 1630. Such a boundary in a velocity image is generated depending on the position of the ultrasonic probe and does not always represent the surface shape of the observation target. For example, when a shading is applied along the boundary, the user may visually recognize it as if the surface of the observation target undulated.

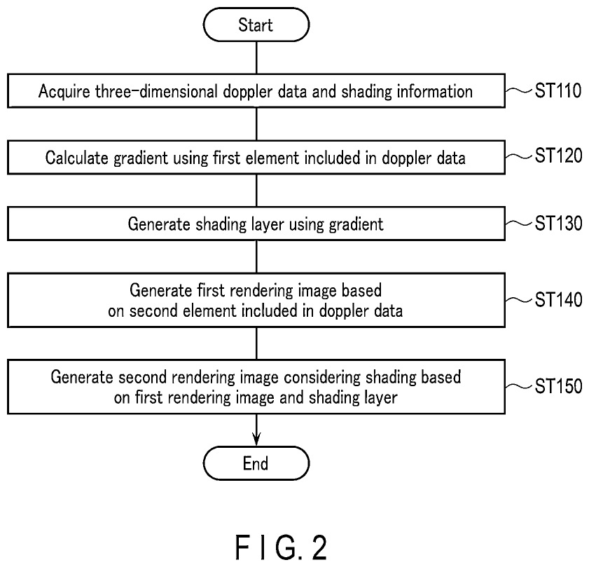

Next, rendering image generation processing according to the first embodiment will be explained. FIG. 2 is a flowchart showing an example of the operation of processing circuitry that executes the rendering image generation processing according to the first embodiment. The rendering image generation processing in FIG. 2 starts when, for example, the user executes a mode in which a rendering image is displayed.

### (Step ST110)

When the rendering image generation processing starts, the processing circuitry 180 executes the acquisition function 185A. By executing the acquisition function 185A, the processing circuitry 180 acquires three-dimensional Doppler data and shading information. The shading information is information of a type of shading (shading type) selected by the user. The information of the shading type includes information of the first element included in the three-dimensional Doppler data used to calculate a gradient, and information of the second element, different from the first element, to generate a basic rendering image.

### (Step ST120)

After acquiring the three-dimensional Doppler data and the shading information, the processing circuitry 180 executes the gradient calculation function 185B. By executing the gradient calculation function 185B, the processing circuitry 180 calculates a gradient using the first element included in the three-dimensional Doppler data. Specifically, the processing circuitry 180 acquires the information of the first element based on the shading information, and uses the first element to calculate the gradient. Note that the processing in step ST120 may be called gradient calculation processing.

### (Step ST130)

After calculating the gradient, the processing circuitry 180 executes the layer generation function 185C. By executing the layer generation function 185C, the processing circuitry 180 generates a shading layer using the calculated gradient. Note that the processing in step ST130 may be called layer generation processing.

### (Step ST140)

After generating the shading layer, the processing circuitry 180 executes the rendering function 185D. By executing the rendering function 185D, the processing circuitry 180 generates the first rendering image not considering shading based on the second element included in the three-dimensional Doppler data.

### (Step ST150)

After generating the first rendering image, the processing circuitry 180 generates by the rendering function 185D the second rendering image considering shading based on the first rendering image and the shading layer. After the processing in step ST150, the rendering image generation processing ends. Note that the processing in step ST140 and step ST150 may be called rendering processing.

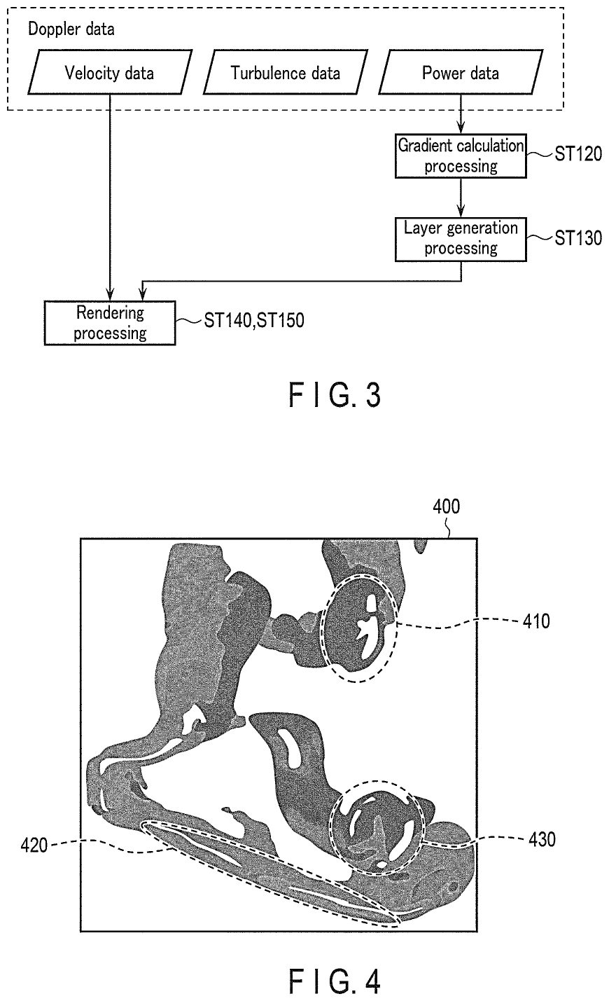

Next, the first concrete example of the rendering image generation processing according to the first embodiment will be explained with reference to FIG. 3. In the first concrete example according to the first embodiment, the power value (power data) of Doppler data is used as the first element, and the velocity value (velocity data) of the Doppler data is used as the second element.

FIG. 3 is a block diagram for explaining the first concrete example of the rendering image generation processing according to the first embodiment. In FIG. 3, the processing circuitry 180 executes the gradient calculation processing (step ST120) and the layer generation processing (step ST130) using power data, and executes the rendering processing (step ST140 and step ST150) using a shading layer based on the power data, and velocity data. A rendering image (second rendering image) considering shading, which is generated by the rendering processing, will be explained with reference to FIG. 4.

FIG. 4 shows an example of a rendering image considering shading according to the first embodiment. A rendering image 400 in FIG. 4 is obtained by superposing a shading layer generated using power data on a rendering image (for example, the rendering image 1500 in FIG. 15) generated using velocity data.

In the rendering image 400, an area 410, an area 420, and an area 430 correspond to the area 1510, the area 1520, and the area 1530 in the rendering image 1500. Shadings (highlights) conforming to shapes of the observation target are applied to the areas 410, 420, and 430. For example, a comparison between the area 430 of the rendering image 400 and the area 1630 of the rendering image 1600 reveals that the shadings correspond to elements for creating shading layers respectively superposed on the rendering image 400 and the rendering image 1600.

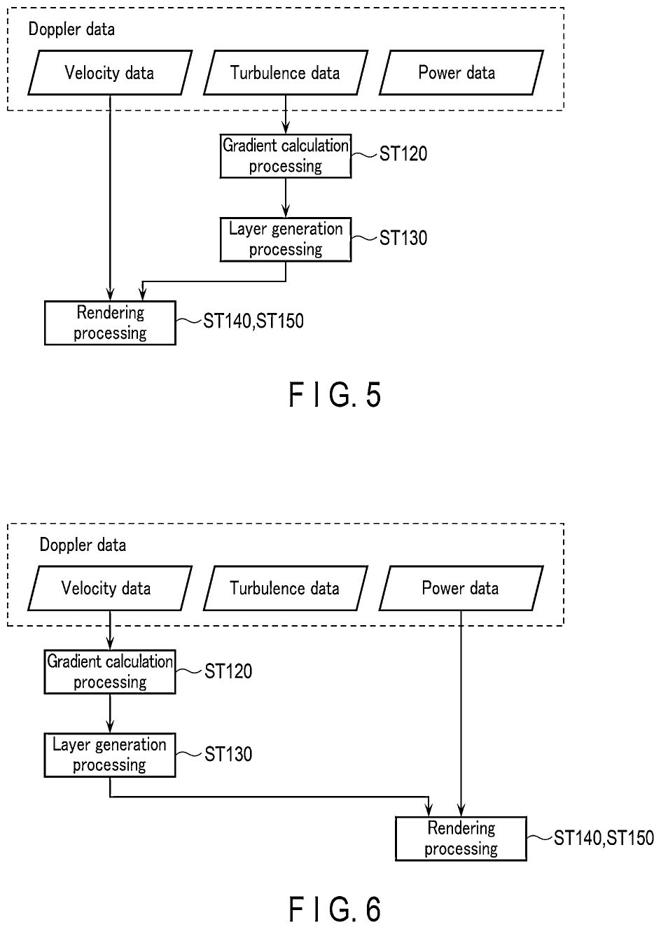

Next, the second concrete example of the rendering image generation processing according to the first embodiment will be explained with reference to FIG. 5. In the second concrete example according to the first embodiment, the turbulence value (turbulence data) of Doppler data is used as the first element, and the velocity value (velocity data) of the Doppler data is used as the second element.

FIG. 5 is a block diagram for explaining the second concrete example of the rendering image generation processing according to the first embodiment. In FIG. 5, the processing circuitry 180 executes the gradient calculation processing (step ST120) and the layer generation processing (step ST130) using turbulence data, and executes the rendering processing (step ST140 and step ST150) using a shading layer based on the turbulence data, and velocity data.

Next, the third concrete example of the rendering image generation processing according to the first embodiment will be explained with reference to FIG. 6. In the third concrete example according to the first embodiment, the velocity value (velocity data) of Doppler data is used as the first element, and the power value (power data) of the Doppler data is used as the second element.

FIG. 6 is a block diagram for explaining the third concrete example of the rendering image generation processing according to the first embodiment. In FIG. 6, the processing circuitry 180 executes the gradient calculation processing (step ST120) and the layer generation processing (step ST130) using velocity data, and executes the rendering processing (step ST140 and step ST150) using a shading layer based on the velocity data, and power data.

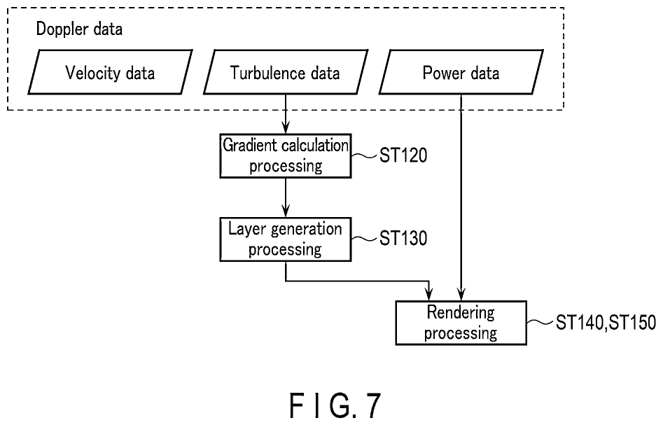

Next, the fourth concrete example of the rendering image generation processing according to the first embodiment will be explained with reference to FIG. 7. In the fourth concrete example according to the first embodiment, the turbulence value (turbulence data) of Doppler data is used as the first element, and the power value (power data) of the Doppler data is used as the second element.

FIG. 7 is a block diagram for explaining the fourth concrete example of the rendering image generation processing according to the first embodiment. In FIG. 7, the processing circuitry 180 executes the gradient calculation processing (step ST120) and the layer generation processing (step ST130) using turbulence data, and executes the rendering processing (step ST140 and step ST150) using a shading layer based on the turbulence data, and power data.

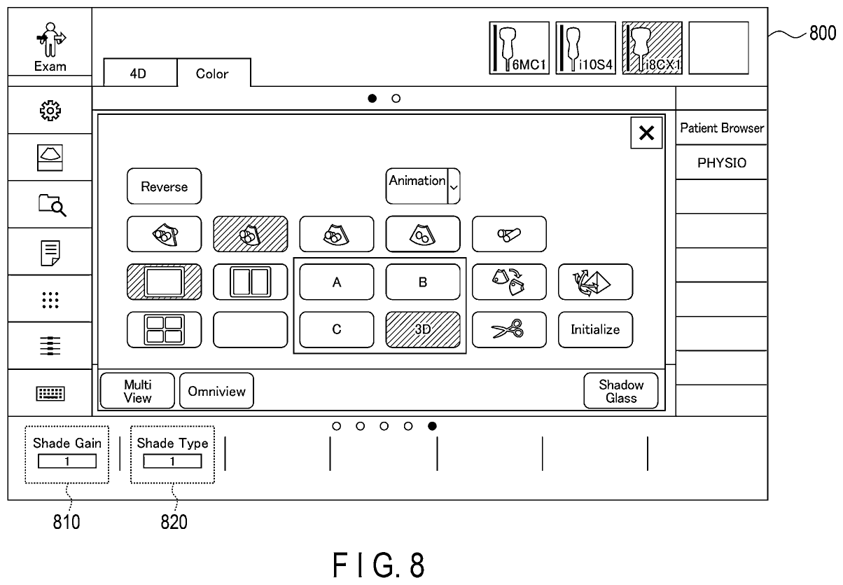

Next, a display image for which the rendering image generation processing is set will be explained with reference to FIG. 8.

FIG. 8 is a view exemplifying a display image for which the rendering image generation processing according to the first embodiment is set. A display image 800 in FIG. 8 is displayed on, for example, the touch panel of the input device 102. A software button 810 for setting a shading gain, and a software button 820 for setting a shading type are displayed on the display image 800.

The shading gain is set by, for example, selecting by the user a shading intensity allocated to each numerical value displayed on the software button 810. The processing circuitry 180 acquires information of a shading intensity selected by the user, and reflects it in a rendering image.

The shading type is set by, for example, selecting by the user a shading type allocated to each numerical value displayed on the software button 820. The shading type includes the conventional example in which the first and second elements are velocity data, velocity+turbulence data, or power data, and the above-described first to fourth concrete examples in which the first and second elements are different pieces of data. The processing circuitry 180 acquires information (shading information) of a shading type selected by the user, and uses the acquired shading information to create a shading layer. Note that the processing circuitry 180 may create a shading layer every time it acquires shading information.

As described above, the ultrasonic diagnosis apparatus according to the first embodiment acquires three-dimensional Doppler data of an observation target, and calculates the gradient of the surface of the observation target using the first element included in the Doppler data. Then, the ultrasonic diagnosis apparatus generates the first rendering image of the observation target based on the second element included in the Doppler data, which is different from the first element, and generates the second rendering image considering shading based on the gradient and the first rendering image.

The ultrasonic diagnosis apparatus according to the first embodiment can generate a basic rendering image and a shading from different elements included in Doppler data, and can increase variations of shading in a rendering image using three-dimensional Doppler data.

### Second Embodiment

The ultrasonic diagnosis apparatus according to the first embodiment generates a shading layer using one element included in Doppler data. In contrast, an ultrasonic diagnosis apparatus according to the second embodiment generates a shading layer using a plurality of elements included in Doppler data.

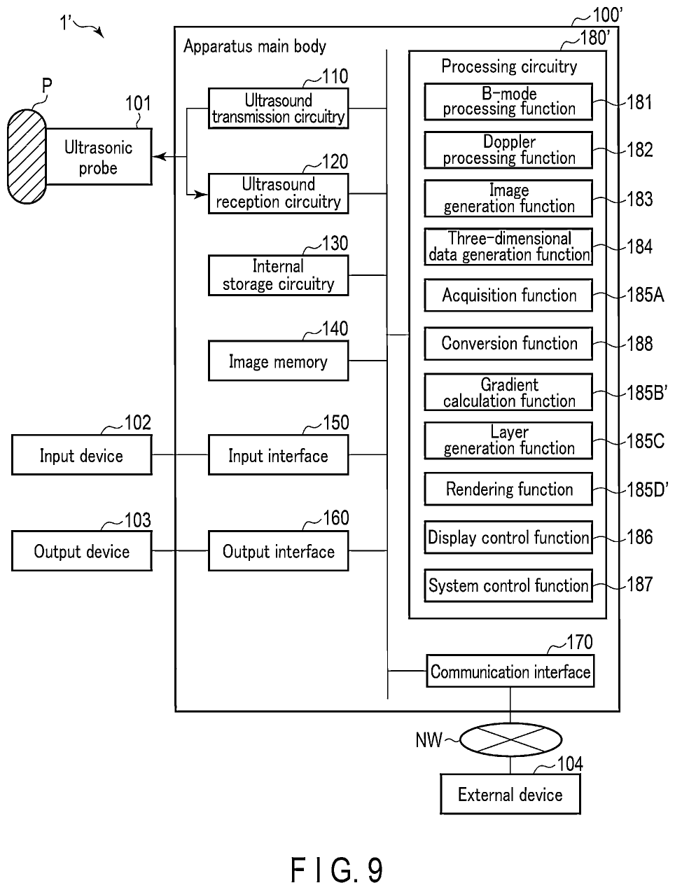

FIG. 9 is a block diagram showing a configuration example of the ultrasonic diagnosis apparatus according to the second embodiment. An ultrasonic diagnosis apparatus 1′ shown in FIG. 9 includes an apparatus main body 100′ and an ultrasonic probe 101. The apparatus main body 100′ is connected to an input device 102 and an output device 103. The apparatus main body 100′ is connected to an external device 104 via a network NW. The external device 104 is a server including, for example, PACS. Note that a description of components having reference numerals similar to those in the first embodiment may be omitted.

The apparatus main body 100′ is an apparatus that generates an ultrasonic image based on a reflection wave signal received by the ultrasonic probe 101. The apparatus main body 100′ includes ultrasound transmission circuitry 110, ultrasound reception circuitry 120, internal storage circuitry 130, an image memory 140, an input interface 150, an output interface 160, a communication interface 170, and processing circuitry 180′.

The processing circuitry 180′ is a processor acting as, for example, a nerve center of the ultrasonic diagnosis apparatus 1′. The processing circuitry 180′ executes programs stored in the internal storage circuitry 130, thereby realizing the functions corresponding to the programs. The processing circuitry 180′ includes, for example, a B-mode processing function 181, a Doppler processing function 182, an image generation function 183, a three-dimensional data generation function 184 functioning as a three-dimensional data generator, an acquisition function 185A functioning as an acquirer, a conversion function 188 functioning as a converter, a gradient calculation function 185B′ functioning as a gradient calculator, a layer generation function 185C functioning as a layer generator, a rendering function 185D′ functioning as a renderer, a display control function 186 functioning as a display controller, and a system control function 187.

The conversion function 188 is a function of converting two different elements (first and second elements) into new elements. In this embodiment, the new elements will be called “converted elements”. By the conversion function 188, the processing circuitry 180′ converts the first and second elements included in three-dimensional Doppler data using predetermined functions, thereby generating converted elements. Hereinafter, a case where the first element is velocity data and the second element is turbulence data will be explained as a concrete example.

Since the turbulence is an index of variations of the velocity, velocity data and turbulence data are considered to be relevant to each other. As a phase volume considering the two pieces of data, a first converted element X and a second converted element Y may be calculated using equations (2) and (3) below:

$$X ( x , y , z ) = g ( T ( x , y , z ) ) cos ( h ( V ( x , y , z ) ) ) \qquad ( 2 )$$

$$Y ( x , y , z ) = g ( T ( x , y , z ) ) sin ( h ( V ( x , y , z ) ) ) \qquad ( 3 )$$

In equation (2), T(x, y, z) represents a turbulence element in a target voxel of three-dimensional Doppler data, and V(x, y, z) represents a velocity element in the target voxel. g( ) is an arbitrary function whose variable is T(x, y, z), and h( ) is an arbitrary function whose variable is V(x, y, z). Note that which of the first converted element X and the second converted element Y is calculated can be selected freely. Hereinafter, either of the first converted element X and the second converted element Y is calculated and simply referred to as “the converted element”.

The gradient calculation function 185B′ is a function of calculating the gradient of the surface of an observation target based on two different elements (first and second elements) included in three-dimensional Doppler data. By the gradient calculation function 185B′, the processing circuitry 180′ calculates the gradient of the surface of the observation target based on the first and second elements included in the three-dimensional Doppler data. Specifically, the processing circuitry 180′ calculates the gradient using elements converted from the first and second elements. Alternatively, the processing circuitry 180′ may calculate the gradient of the surface of a blood vessel using the velocity value and turbulence value of the Doppler data.

The rendering function 185D′ is a function of generating a rendering image considering shading. By the rendering function 185D′, the processing circuitry 180′ generates the basic rendering image (first rendering image) of the observation target based on an element (third element) different from at least either of the first and second elements used to generate converted elements. Based on the calculated gradient and the first rendering image, the processing circuitry 180′ generates the second rendering image considering shading.

Specifically, the processing circuitry 180′ generates the second rendering image based on the first rendering image and a shading layer generated from the calculated gradient. More specifically, the processing circuitry 180′ generates the second rendering image by superposing the shading layer on the first rendering image.

The configuration of the ultrasonic diagnosis apparatus according to the second embodiment has been described above. Next, rendering image generation processing according to the second embodiment will be explained.

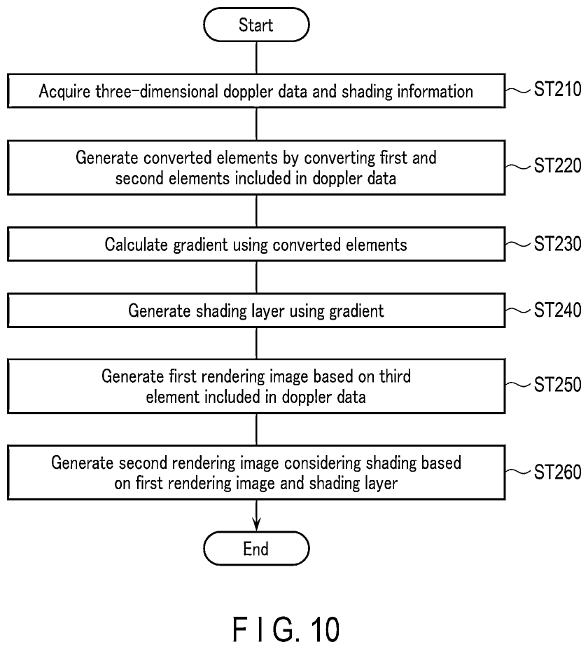

FIG. 10 is a flowchart showing an example of the operation of processing circuitry that executes the rendering image generation processing according to the second embodiment. The rendering image generation processing in FIG. 10 starts when, for example, the user executes a mode in which a rendering image is displayed.

### (Step ST210)

When the rendering image generation processing starts, the processing circuitry 180′ executes the acquisition function 185A. By executing the acquisition function 185A, the processing circuitry 180′ acquires three-dimensional Doppler data and shading information. The shading information is information of a type of shading (shading type) selected by the user. The information of the shading type in the second embodiment includes information of the first and second elements included in the three-dimensional Doppler data used to calculate a gradient, and information of the third element to generate a basic rendering image. Note that the third element may be the same as either of the first and second elements or different from the first and second elements.

### (Step ST220)

After acquiring the three-dimensional Doppler data and the shading information, the processing circuitry 180′ executes the conversion function 188. By executing the conversion function 188, the processing circuitry 180′ generates converted elements by converting the first and second elements included in the three-dimensional Doppler data. Note that the processing in step ST220 may be called conversion processing.

### (Step ST230)

After generating the converted elements, the processing circuitry 180′ executes the gradient calculation function 185B′. By executing the gradient calculation function 185B′, the processing circuitry 180′ calculates a gradient using the converted elements. Note that the processing in step ST230 may be called gradient calculation processing.

### (Step ST240)

After calculating the gradient, the processing circuitry 180′ executes the layer generation function 185C. By executing the layer generation function 185C, the processing circuitry 180′ generates a shading layer using the calculated gradient. Note that the processing in step ST240 may be called layer generation processing.

### (Step ST250)

After generating the shading layer, the processing circuitry 180′ executes the rendering function 185D′. By executing the rendering function 185D′, the processing circuitry 180′ generates the first rendering image not considering shading based on the third element included in the three-dimensional Doppler data.

### (Step ST260)

After generating the first rendering image, the processing circuitry 180′ generates by the rendering function 185D′ the second rendering image considering shading based on the first rendering image and the shading layer. After the processing in step ST260, the rendering image generation processing ends. Note that the processing in step ST250 and step ST260 may be called rendering processing.

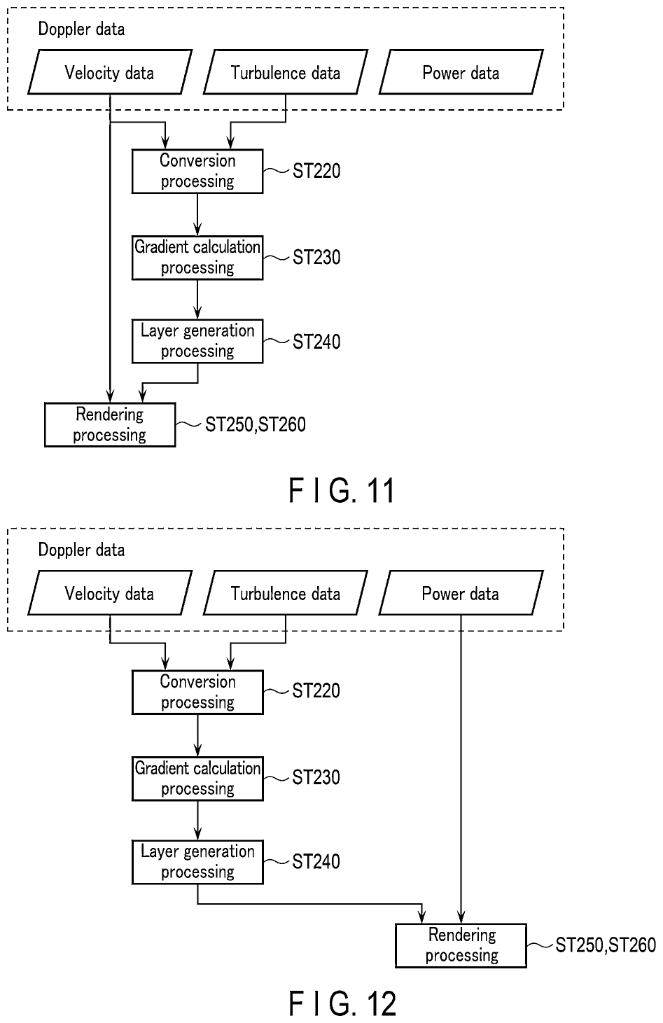

Next, the first concrete example of the rendering image generation processing according to the second embodiment will be explained with reference to FIG. 11. In the first concrete example according to the second embodiment, the velocity value (velocity data) of Doppler data is used as the first and third elements, and the turbulence value (turbulence data) of the Doppler data is used as the second element.

FIG. 11 is a block diagram for explaining the first concrete example of the rendering image generation processing according to the second embodiment. In FIG. 11, the processing circuitry 180′ executes the conversion processing (step ST220), the gradient calculation processing (step ST230), and the layer generation processing (step ST240) using velocity data and turbulence data, and executes the rendering processing (step ST250 and step ST260) using a shading layer based on the converted data, and velocity data.

Next, the second concrete example of the rendering image generation processing according to the second embodiment will be explained with reference to FIG. 12. In the second concrete example according to the second embodiment, the velocity value (velocity data) of Doppler data is used as the first element, the turbulence value (turbulence data) of the Doppler data is used as the second element, and the power value (power data) of the Doppler data is used as the third element.

FIG. 12 is a block diagram for explaining the second concrete example of the rendering image generation processing according to the second embodiment. In FIG. 12, the processing circuitry 180′ executes the conversion processing (step ST220), the gradient calculation processing (step ST230), and the layer generation processing (step ST240) using velocity data and turbulence data, and executes the rendering processing (step ST250 and step ST260) using a shading layer based on the converted data, and velocity data.

As described above, the ultrasonic diagnosis apparatus according to the second embodiment acquires three-dimensional Doppler data for an observation target, and calculates the gradient of the surface of the observation target based on the first and second elements included in the Doppler data. Then, the ultrasonic diagnosis apparatus generates the first rendering image of the observation target based on the third element included in the Doppler data, which is different from the second element, and generates the second rendering image considering shading based on the gradient and the first rendering image. Alternatively, the ultrasonic diagnosis apparatus may generate converted elements by converting the first and second elements using predetermined functions, and calculate the gradient using the converted elements.

Similar to the first embodiment, the ultrasonic diagnosis apparatus according to the second embodiment can increase variations of shading in a rendering image using three-dimensional Doppler data.

### Third Embodiment

In the first and second embodiments, the ultrasonic diagnosis apparatus having a plurality of functions regarding rendering image generation processing has been described. To the contrary, in the third embodiment, an information processing apparatus having a plurality of functions corresponding to the first embodiment will be explained.

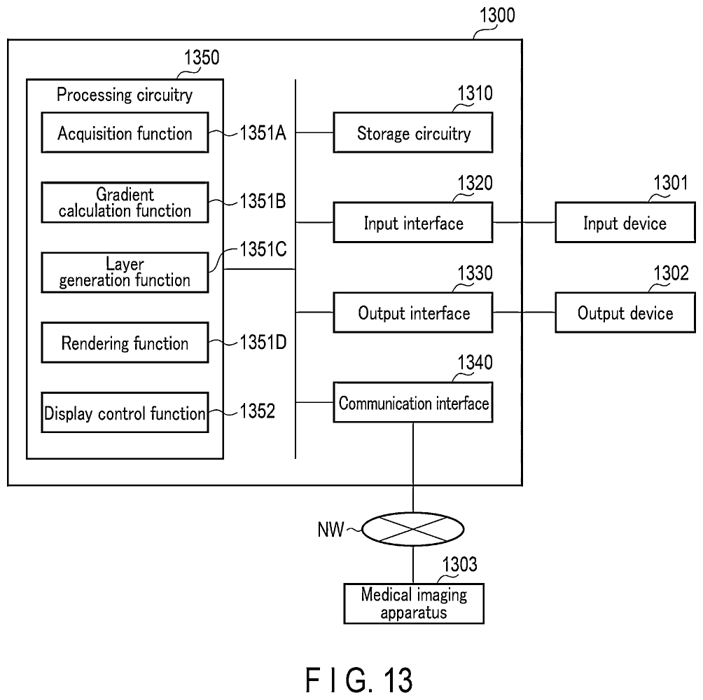

FIG. 13 is a block diagram showing a configuration example of the information processing apparatus according to the third embodiment. An information processing apparatus 1300 shown in FIG. 13 is connected to an input device 1301 and an output device 1302. The information processing apparatus 1300 is also connected to a medical imaging apparatus 1303 via a network NW. The medical imaging apparatus 1303 corresponds to, for example, an ultrasonic diagnosis apparatus. The input device 1301 and the output device 1302 are substantially the same as the input device 102 and the output device 103 shown in FIG. 1.

The information processing apparatus 1300 is an apparatus that performs the rendering image generation processing to generate a rendering image. The information processing apparatus 1300 includes storage circuitry 1310, an input interface 1320, an output interface 1330, a communication interface 1340, and processing circuitry 1350.

The storage circuitry 1310 includes a processor-readable storage medium, such as a magnetic storage medium, an optical storage medium, or a semiconductor memory. The storage circuitry 1310 stores programs relating to the rendering image generation processing, various data, etc. The programs and various data may be pre-stored in the storage circuitry 1310. Alternatively, the programs and various data may be stored and distributed in a non-transitory storage medium, read from the non-transitory storage medium and installed in the storage circuitry 1310.

The storage circuitry 1310 stores B-mode image data, contrast image data, image data relating to bloodstream visualization, three-dimensional data, etc. generated at the medical imaging apparatus 1303, in accordance with an operation that is input via the input interface 1320.

The storage circuitry 1310 may be a drive etc. which reads and writes various types of information to and from a portable storage medium, such as a CD drive, a DVD drive, and a flash memory. The storage circuitry 1310 may write the stored data onto a portable storage medium to store the data into an external device by way of the portable storage medium.

The input interface 1320 receives various instructions from the operator through the input device 1301. The input device 1301 is, for example, a mouse, a keyboard, a panel switch, a slider switch, a trackball, a rotary encoder, an operation panel, or a touch panel. The input interface 1320 is connected to the processing circuitry 1350 via a bus, for example, so that it can convert an operation instruction that is input by the operator into an electric signal, and output the electric signal to the processing circuitry 1350. The input interface 1320 is not limited to physical operation components such as a mouse and a keyboard. Examples of the input interface may include a circuit configured to receive an electric signal corresponding to an operation instruction that is input from an external input device provided separately from the information processing apparatus 1300 and to output this electric signal to the processing circuitry 1350.

The output interface 1330 is an interface to output, for example, the electric signal from the processing circuitry 1350 to the output device 1302. The output device 1302 may be any display such as a liquid crystal display, an organic EL display, an LED display, a plasma display, or a CRT display. The output device 1302 may be a touch-panel display that also serves as the input device 1301. The output device 1302 may also include a speaker configured to output a voice in addition to the display. The output interface 1330 is connected to the processing circuitry 1350, for example, via a bus, and outputs the electric signal from the processing circuitry 1350 to the output device 1302.

The communication interface 1340 is connected to the medical imaging apparatus 1303 via, for example, the network NW, and performs data communication with the medical imaging apparatus 1303.

The processing circuitry 1350 is a processor acting as a nerve center of the information processing apparatus 1300, for example. The processing circuitry 1350 executes the programs stored in the storage circuitry 1310, thereby realizing the functions corresponding to the programs. The processing circuitry 1350 includes, for example, an acquisition function 1351A functioning as an acquirer, a gradient calculation function 1351B functioning as a gradient calculator, a layer generation function 1351C functioning as a layer generator, a rendering function 1351D functioning as a renderer, and a display control function 1352 functioning as a display controller.

The acquisition function 1351A is a function of acquiring data about rendering image generation processing. By the acquisition function 1351A, the processing circuitry 1350 acquires three-dimensional Doppler data of an observation target.

The gradient calculation function 1351B is a function of calculating the gradient of the surface of an observation target using the first element included in three-dimensional Doppler data. By the gradient calculation function 1351B, the processing circuitry 1350 calculates the gradient of the surface of the observation target using the first element included in the three-dimensional Doppler data.

The layer generation function 1351C is a function of generating a shading layer using the gradient of a voxel corresponding to the surface of an observation target. By the layer generation function 1351C, the processing circuitry 1350 generates a shading layer from the gradient using a known shading model.

The rendering function 1351D is a function of generating a rendering image. By the rendering function 1351D, the processing circuitry 1350 generates the first rendering image of the observation target based on the second element included in the three-dimensional Doppler data, which is different from the first element used for gradient calculation, and generates the second rendering image considering shading based on the calculated gradient and the first rendering image.

Specifically, the processing circuitry 1350 generates the second rendering image based on the first rendering image and a shading layer generated from the calculated gradient. More specifically, the processing circuitry 1350 generates the second rendering image by superposing the shading layer on the first rendering image.

The display control function 1352 is a function of displaying a rendering image. By the display control function 1352, the processing circuitry 1350 generates the second rendering image. Alternatively, the processing circuitry 1350 may switch between the first rendering image and the second rendering image. Note that the processing circuitry 1350 may display an interface that allows the user to select a shading type.

Note that the information processing apparatus 1300 may generate three-dimensional Doppler data based on data (for example, a reception signal in the ultrasonic diagnosis apparatus) relating to a living body that is received from the medical imaging apparatus 1303.

A combination of the first and second elements in the third embodiment may be the same as a combination described in each concrete example of the first embodiment.

Therefore, the information processing apparatus according to the third embodiment can expect effects similar to those according to the first embodiment.

### Fourth Embodiment

In the first and second embodiments, the ultrasonic diagnosis apparatus having a plurality of functions regarding rendering image generation processing has been described. In the third embodiment, the information processing apparatus having a plurality of functions corresponding to the first embodiment has been described. In contrast, in the fourth embodiment, an information processing apparatus having a plurality of functions corresponding to the second embodiment will be explained.

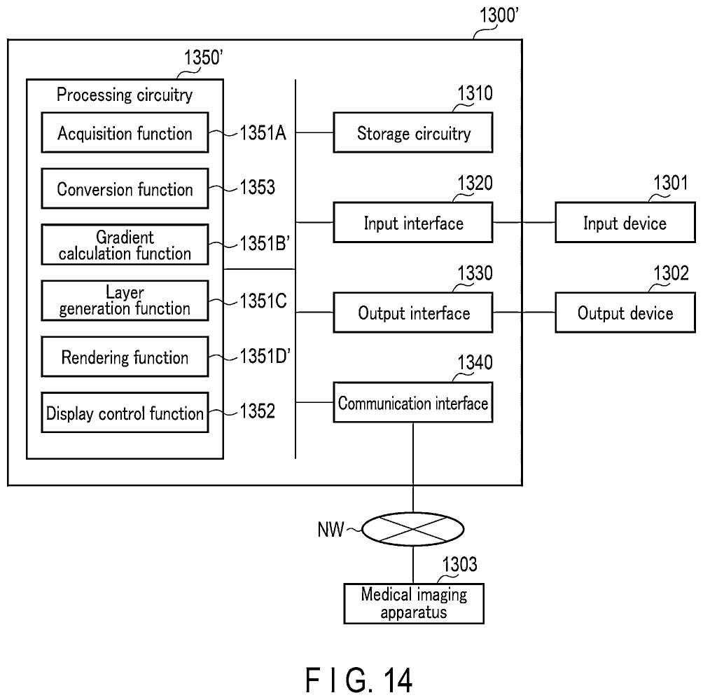

FIG. 14 is a block diagram showing a configuration example of the information processing apparatus according to the fourth embodiment. An information processing apparatus 1300′ shown in FIG. 14 is connected to a medical imaging apparatus 1303 via a network NW. The medical imaging apparatus 1303 corresponds to, for example, an ultrasonic diagnosis apparatus. Note that a description of components having reference numerals similar to those in the third embodiment may be omitted.

The information processing apparatus 1300′ is an apparatus that performs the rendering image generation processing to generate a rendering image. The information processing apparatus 1300′ includes storage circuitry 1310, an input interface 1320, an output interface 1330, a communication interface 1340, and processing circuitry 1350′.

The processing circuitry 1350′ is a processor acting as, for example, a nerve center of the information processing apparatus 1300′. The processing circuitry 1350′ executes programs stored in the storage circuitry 1310, thereby realizing the functions corresponding to the programs. The processing circuitry 1350′ includes, for example, an acquisition function 1351A functioning as an acquirer, a conversion function 1353 functioning as a converter, a gradient calculation function 1351B′ functioning as a gradient calculator, a layer generation function 1351C functioning as a layer generator, a rendering function 1351D′ functioning as a renderer, and a display control function 1352 functioning as a display controller.

The conversion function 1353 is a function of converting two different elements (first and second elements) into new elements (converted elements). By the conversion function 1353, the processing circuitry 1350′ converts the first and second elements included in three-dimensional Doppler data using predetermined functions, thereby generating converted elements.

The gradient calculation function 1351B′ is a function of calculating the gradient of the surface of an observation target based on two different elements (first and second elements) included in three-dimensional Doppler data. By the gradient calculation function 1351B′, the processing circuitry 1350′ calculates the gradient of the surface of the observation target based on the first and second elements included in the three-dimensional Doppler data. Specifically, the processing circuitry 1350′ calculates the gradient using elements converted from the first and second elements. Alternatively, the processing circuitry 1350′ may calculate the gradient of the surface of a blood vessel using the velocity value and turbulence value of the Doppler data.

The rendering function 1351D′ is a function of generating a rendering image considering shading. By the rendering function 1351D′, the processing circuitry 1350′ generates the first rendering image of the observation target based on the third element different from at least either of the first and second elements used to generate converted elements. Based on the calculated gradient and the first rendering image, the processing circuitry 1350′ generates the second rendering image considering shading.

Specifically, the processing circuitry 1350′ generates the second rendering image based on the first rendering image and a shading layer generated from the calculated gradient. More specifically, the processing circuitry 1350′ generates the second rendering image by superposing the shading layer on the first rendering image.

Note that the information processing apparatus 1300′ may generate three-dimensional Doppler data based on data (for example, a reception signal in the ultrasonic diagnosis apparatus) relating to a living body that is received from the medical imaging apparatus 1303.

A combination of the first, second, and third elements in the fourth embodiment may be the same as a combination described in each concrete example of the second embodiment.

Therefore, the information processing apparatus according to the fourth embodiment can expect effects similar to those according to the second embodiment.

### Modifications

A specific combination of elements (for example, the first element is power data and the second element is velocity data) included in Doppler data has been described in the above embodiments, but the present invention is not limited to this. For example, a combination of elements included in Doppler data is arbitrary. This can further increase variations of shading.

Rendering processing using elements included in Doppler data has been described in the above embodiments, but the present invention is not limited to this. For example, rendering processing may be performed using data obtained by performing predetermined processing (for example, processing of reducing a motion artifact at a low flow rate) on Doppler data.

Volume rendering (or global illumination) has been described as creation of a rendering image in the above embodiments, but the present invention is not limited to this. For example, as creation of a rendering image, surface rendering that expresses the surface of an object, thickened Multi-Planner Reconstruction (MPR), and Maximum Intensity Projection (MIP) may be used.

According to at least one of the above-described embodiments, variations of shading in a rendering image using three-dimensional Doppler data can be increased.

Note that the term “processor” used in the above description means a circuit such as a Central Processing Unit (CPU), a Graphics Processing Unit (GPU), an Application Specific Integrated Circuit (ASIC), a programmable logic device (for example, a Simple Programmable Logic Device (SPLD)), or a Complex Programmable Logic Device (CPLD), or a Field Programmable Gate Array (FPGA). When the processor is, for example, a CPU, it implements functions by reading out and executing programs stored in the storage circuitry. In contrast, when the processor is, for example, an ASIC, the functions are directly installed as logic circuits in the circuitry of the processor, instead of storing programs in the storage circuitry. Note that each processor in the embodiment may be constituted as a single circuit for each processor, or a plurality of independent circuits may be combined into one processor to implement its functions. Further, a plurality of building components in the drawings may be integrated into one processor to implement its functions.

In addition, each function according to the embodiment can be implemented by installing, in a computer such as a workstation, programs for executing the above-described processing, and loading them on the memory. At this time, programs capable of causing the computer to execute the above-described method can be stored and distributed in a storage medium such as a magnetic disk (for example, a hard disk), an optical disk (for example, a CD-ROM or a DVD), or a semiconductor memory.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnosis apparatus, comprising processing circuitry configured to:
acquire three-dimensional Doppler data of an observation target;
calculate a gradient of a surface of the observation target using a first element included in the Doppler data;
generate a first rendering image of the observation target based on a second element included in the Doppler data, which is different from the first element; and
generate a second rendering image considering shading based on the gradient and the first rendering image.

2. The ultrasonic diagnosis apparatus according to claim 1, wherein the processing circuitry is further configured to:
generate a shading layer to apply the shading to the first rendering image using the gradient; and
generate the second rendering image based on the first rendering image and the shading layer.

3. The ultrasonic diagnosis apparatus according to claim 2, wherein the processing circuitry is further configured to generate the second rendering image by superposing the shading layer on the first rendering image.

4. The ultrasonic diagnosis apparatus according to claim 1, wherein the first element is a power value of the Doppler data, and the second element is a velocity value of the Doppler data.

5. The ultrasonic diagnosis apparatus according to claim 1, wherein the first element is a turbulence value of the Doppler data, and the second element is a velocity value of the Doppler data.

6. The ultrasonic diagnosis apparatus according to claim 1, wherein the first element is a velocity value of the Doppler data, and the second element is a power value of the Doppler data.

7. The ultrasonic diagnosis apparatus according to claim 1, wherein the first element is a turbulence value of the Doppler data, and the second element is a power value of the Doppler data.

8. The ultrasonic diagnosis apparatus according to claim 1, wherein the processing circuitry is further configured to:
display an interface that allows a user to select a type of the shading; and
acquire shading information representing the type of the shading selected by the user,
wherein the shading information includes a combination of the first element and the second element.

9. An ultrasonic diagnosis apparatus, comprising processing circuitry configured to:
acquire three-dimensional Doppler data of an observation target;
calculate a gradient of a surface of the observation target based on a first element and a second element included in the Doppler data;
generate a first rendering image of the observation target based on a third element included in the Doppler data, which is different from the second element; and
generate a second rendering image considering shading based on the gradient and the first rendering image.

10. The ultrasonic diagnosis apparatus according to claim 9, wherein the processing circuitry is further configured to:
generate converted elements by converting the first element and the second element using predetermined functions; and
calculate the gradient using the converted elements.

11. The ultrasonic diagnosis apparatus according to claim 10, wherein the processing circuitry is further configured to:
generate a shading layer to apply the shading to the first rendering image using the gradient; and
generate the second rendering image based on the first rendering image and the shading layer.

12. The ultrasonic diagnosis apparatus according to claim 9, wherein the first element is a velocity value of the Doppler data, the second element is a turbulence value of the Doppler data, and the third element is identical to the first element.

13. The ultrasonic diagnosis apparatus according to claim 9, wherein the first element is a velocity value of the Doppler data, the second element is a turbulence value of the Doppler data, and the third element is a power value of the Doppler data.

14. The ultrasonic diagnosis apparatus according to claim 9, wherein the processing circuitry is further configured to:
display an interface that allows a user to select a type of the shading; and
acquire shading information representing the type of the shading selected by the user,
wherein the shading information includes a combination of the first element, the second element, and the third element.

15. An image processing apparatus, comprising processing circuitry configured to:
acquire three-dimensional Doppler data of an observation target;
calculate a gradient of a surface of the observation target using a first element included in the Doppler data;
generate a first rendering image of the observation target based on a second element included in the Doppler data, which is different from the first element; and
generate a second rendering image considering shading based on the gradient and the first rendering image.

16. An image processing apparatus, comprising processing circuitry configured to:
acquire three-dimensional Doppler data of an observation target;
calculate a gradient of a surface of the observation target based on a first element and a second element included in the Doppler data;
generate a first rendering image of the observation target based on a third element included in the Doppler data, which is different from the second element; and
generate a second rendering image considering shading based on the gradient and the first rendering image.

17. An image processing method, comprising:
acquiring three-dimensional Doppler data of an observation target;
calculating a gradient of a surface of the observation target using a first element included in the Doppler data;
generating a first rendering image of the observation target based on a second element included in the Doppler data, which is different from the first element; and
generating a second rendering image considering shading based on the gradient and the first rendering image.

18. An image processing method, comprising:
acquiring three-dimensional Doppler data of an observation target;
calculating a gradient of a surface of the observation target based on a first element and a second element included in the Doppler data;
generating a first rendering image of the observation target based on a third element included in the Doppler data, which is different from the second element; and
generating a second rendering image considering shading based on the gradient and the first rendering image.
